(19) United States
(12) Patent Application Publication
(10) Pub. No.: US 2011/0245562 A1
(43) Pub. Date: Oct. 6, 2011

(54) Pulsed oxidative dehydrogenation process

(75) Inventors: Leonid Modestovich Kustov, Moscow (RU); Aleksey Victorovich Kucherov, Moscow (RU); Elena Dmitrievna Finashina, Moscow (RU); Alexander Yurievich Stakheev, Moscow (RU); Ilya Mikhailovich Sinev, Moscow (RU); Andrzej Krzywicki, Calgary (CA)

(73) Assignee: NOVA Chemicals (International) S.A.

(21) Appl. No.: 13/065,250

(22) Filed: Mar. 17, 2011

(30) Foreign Application Priority Data
Mar. 31, 2010 (CA) 2701089

(51) Int. Cl. C07C 5/333

(52) U.S. Cl. 585/443; 585/658

(57) ABSTRACT

The present invention provides a continuous process for the oxidative dehydrogenation of a lower paraffin to a lower olefin, preferably alpha olefin by sequentially providing pulses of an oxygen containing gas, an inert gas, the paraffin, and inert gas in the presence of a catalyst that preferably has the ability to hold and release oxygen, so that the paraffin and the oxygen do not directly mix in the reactor.

### FIELD OF THE INVENTION

The present invention relates to the oxidative dehydrogenation of paraffins and ethyl benzene to corresponding olefins and styrene. More particularly the present invention relates to a pulsed process for the oxidative dehydrogenation of paraffins and ethyl benzene wherein the catalytic bed comprises at least one component which extracts oxygen from an oxygen containing gas and releases oxygen to the oxidative dehydrogenation and pulses of hydrocarbyl feed selected from the group consisting of paraffin and ethyl benzene and pulses of an oxygen containing gas are separated by a pulse of inert gas sufficiently big to prevent the oxygen and hydrocarbyl feed components from mixing.

### BACKGROUND OF THE INVENTION

The thermal cracking of paraffins to olefins, particularly lower paraffins such as C₂₋₄ paraffins typically ethane and propane to corresponding olefins is an energy intensive process. Currently paraffins, particularly aliphatic paraffins, are converted to olefins using thermal cracking technology. Typically the paraffins are passed through a furnace tube heated to at least 800° C., typically from about 850° C. to the upper working temperature of the alloy for the furnace tube, generally about 950° C. to 1000° C., for a period of time in the order of milliseconds to a few seconds. The paraffin molecule loses hydrogen and one or more unsaturated bonds are formed to produce an olefin. The current thermal cracking processes are not only cost intensive to build and operate but also energy intensive due to the substantial heat requirement for the endothermic cracking reactions. As a result, significant amounts of CO₂ are produced from the operation of these cracking furnaces.

Dehydrogenation processes are widely used in modern refining and petrochemistry. Processes for the synthesis of butadiene, isoprene, and long-chain olefins are commercialized. However, the area of dehydrogenation of light alkanes remains to be underexplored and especially ethane dehydrogenation is far from the commercial scale. The most advanced are the processes of oxidative dehydrogenation based on the use of transition metal oxide catalysts and a robust oxidant, such as oxygen or air. The oxidative conversion makes the process of dehydrogenation thermodynamically advantageous and decreases the reaction temperature as compared to non-oxidative processes (e.g. thermal cracking). The conversion of ethane, which is the second major component of natural gas, to ethylene requires development of new processes.

Alternatively, it is known that olefins can be produced by reactions between paraffins with oxygen. However, this technology has not been commercially practiced for a number of reasons including the potential for an explosive mixture of oxygen and paraffin at an elevated temperature. For satisfactory conversion of paraffins to olefins, the required oxygen in the feed mixture should be typically higher than the maximum allowable level before entering the explosion range. Another reason is the requirement of either front end oxygen separation (from air) or a back end nitrogen separation, which often brings the overall process economy into negative territory. Therefore, solutions to address these issues are being sorted in various directions.

In the current prior art when a mixed feed of oxygen and hydrocarbon is used care must be taken so that the amount of oxygen in the mixture does not exceed about 25% or the mixed feed will exceed an explosive limit. As far as applicants have been able to determine none of the prior art in this field suggests the pulsed feed approach to segregate the hydrocarbon feed from the oxygen containing feed to minimize the potential for a mixture of oxygen and hydrocarbon to occur or if such mixture occurs to approach the explosive limit.

There are a number of United States patents assigned to Petro-Tex Chemical Corporation issued in the late 1960's that disclose the use of various ferrites in a steam cracker to produce olefins from paraffins. The patents include U.S. Pat. Nos. 3,420,911 and 3,420,912 in the names of Woskow et al. The patents teach introducing ferrites such as zinc, cadmium, and manganese ferrites (i.e. mixed oxides with iron oxide). The ferrites are introduced into a dehydrogenation zone at a temperature from about 250° C. up to about 750° C. at pressures less than 100 psi (689.476 kPa) for a time less than 2 seconds, typically from 0.005 to 0.9 seconds. However the reaction does not take place in the presence of a catalyst of the type of the present invention.

In the Petro-Tex patents the metal ferrite (e.g. MFeO₄ where, for example, M is Mg, Mn, Co, Ni, Zn or Cd) is circulated through the dehydrogenation zone and then to a regeneration zone where the ferrite is reoxidized and then fed back to the dehydrogenation zone.

The patent GB 1,213,181, which seems to correspond in part to the above Petro-Tex patents, discloses that nickel ferrite may be used in the oxidative dehydrogenation process. The reaction conditions are comparable to those of above noted Petro-Tex patents.

Subsequent to the Petro-Tex patents a number of patents were published relating to the catalytic dehydrogenation of paraffins. However, these patents do not include the use of the ferrites of the Petro-Tex patents to provide a source of oxygen.

Several catalytic systems are known in the art for the oxidative dehydrogenation of ethane. U.S. Pat. No. 4,450,313, issued May 22, 1984 to Eastman et al., assigned to Phillips Petroleum Company discloses a catalyst of the composition LiO—TiO₂, which is characterized by a low ethane conversion not exceeding 10%, in spite of a rather high selectivity to ethylene (92%). The major drawback of this catalyst is the high temperature of the process of oxidative dehydrogenation, which is close to or higher than 650° C.

The U.S. Pat. No. 6,624,116, issued Sep. 23, 2003 to Bharadwaj et al. and U.S. Pat. No. 6,566,573 issued May 20, 2003 to Bharadwaj et al., both assigned to Dow Global Technologies Inc., disclose Pt—Sn—Sb—Cu—Ag monolith systems that have been tested in an autothermal regime at T>750° C., the starting gas mixture contained hydrogen (H₂:O₂=2:1, GHSV=180 000 h⁻¹). The catalyst composition is different from that of the present invention and the present invention does not contemplate the use of molecular hydrogen in the feed.

U.S. Pat. No. 4,524,236 issued Jun. 18, 1985 to McCain, assigned to Union Carbide Corporation and U.S. Pat. No. 4,899,003 issued Feb. 6, 1990 to Manyik et al., assigned to Union Carbide Chemicals and Plastics Company Inc. disclose mixed metal oxide catalysts of V—Mo—Nb—Sb. At 375-400° C. the ethane conversion reached 70% with the selectivity close to 71-73%. However, these parameters were achieved only at very low gas hourly space velocities less than 900 h⁻¹ (i.e. 720 h⁻¹).

Rather promising results were obtained for nickel-containing catalysts disclosed in U.S. Pat. No. 6,891,075 issued May 10, 2005 to Liu, assigned to Symyx Technologies Inc. At 325° C. the ethane conversion on the best catalyst in this series was about 20% with a selectivity of 85% (a Ni—Nb—Ta oxide catalyst). The patent teaches a catalyst for the oxidative dehydrogenation of a paraffin (alkane) such as ethane. The gaseous feedstock comprises at least the alkane and oxygen, but may also include diluents (such as argon, nitrogen, etc.) or other components (such as water or carbon dioxide). The dehydrogenation catalyst comprises at least about 2 weight % of NiO and a broad range of other elements preferably Nb, Ta, and Co. While NiO is present in the catalyst it does not appear to be the source of the oxygen for the oxidative dehydrogenation of the alkane (ethane).

U.S. Pat. No. 6,521,808 issued Feb. 18, 2003 to Ozkan et al., assigned to the Ohio State University, teaches sol gel supported catalysts for the oxidative dehydrogenation of ethane to ethylene. The catalyst appears to be a mixed metal system such as Ni—Co—Mo, V—Nb—Mo possibly doped with small amounts of Li, Na, K, Rb and Cs on a mixed silica oxide/titanium oxide support. Again the catalyst does not provide the oxygen for the oxidative dehydrogenation rather gaseous oxygen is included in the feed.

U.S. Pat. No. 7,319,179 issued Jan. 15, 2008 to Lopez-Nieto et al., assigned to Consejo Superior de Investigaciones Cientificas and Universidad Politecnica de Valencia, discloses Mo—V—Te—Nb—O oxide catalysts that provided an ethane conversion of 50-70% and selectivity to ethylene up to 95% (at 38% conversion) at 360-400° C. The catalysts have the empirical formula MoTeₕVᵢNbⱼAₖOₓ, where A is a fifth modifying element. The catalyst is a calcined mixed oxide (at least of Mo, Te, V and Nb), optionally supported on: (i) silica, alumina and/or titania, preferably silica at 20-70 wt % of the total supported catalyst or (ii) silicon carbide. The supported catalyst is prepared by conventional methods of precipitation from solutions, drying the precipitate then calcining.

Similar catalysts have been also described in open publications of Lopez-Nieto and co-authors. Selective oxidation of short-chain alkanes over hydrothermally prepared MoVTeNbO catalysts is discussed by F. Ivars, P. Botella, A. Dejoz, J. M. Lopez-Nieto, P. Concepcion, and M. I. Vazquez, in Topics in Catalysis (2006), 38(1-3), 59-67.

MoVTe—Nb oxide catalysts have been prepared by a hydrothermal method and tested in the selective oxidation of propane to acrylic acid and in the oxidative dehydrogenation of ethane to ethylene. The influence of the concentration of oxalate anions in the hydrothermal gel has been studied for two series of catalysts, Nb-free and Nb-containing, respectively. Results show that the development of an active and selective active orthorhombic phase (Te₂M₂₀O₅₇, M=Mo, V, Nb) requires an oxalate/Mo molar ratio of 0.4-0.6 in the synthesis gel in both types of samples. The presence of Nb favors a higher catalytic activity in both ethane and propane oxidation and a better production of acrylic acid.

Mixed metal oxide supported catalyst compositions, catalyst manufacture and use in ethane oxidation are described in Patent WO 2005018804 A1, 3 Mar. 2005, assigned to BP Chemicals Limited, UK. A catalyst composition for the oxidation of ethane to ethylene and acetic acid comprises (i) a support and (ii) in combination with O, the elements Mo, V and Nb, optionally W and a component Z, which is metals of Group 14. Thus, Mo_{60.5}V₃₂Nb_{7.5}Oₓ on silica was modified with 0.33 g-atom ratio Sn for ethane oxidation with good ethylene/acetic acid selectivity and product ratio 1:1.

A process for preparation of ethylene from gaseous feed comprising ethane and oxygen involving contacting the feed with a mixed oxide catalyst containing vanadium, molybdenum, tantalum and tellurium in a reactor to form an effluent of ethylene is disclosed in WO 2006130288 A1, 7 Dec. 2006, assigned to Celanese Int. Corp. The catalyst has a selectivity for ethylene of 50-80% thereby allowing oxidation of ethane to produce ethylene and acetic acid with high selectivity. The catalyst has the formula Mo₁V_{0.3}Ta_{0.1}Te_{0.3}O_{z}. The catalyst is optionally supported on a support selected from porous silicon dioxide, fused silica, kieselguhr, silica gel, porous and nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, aluminum silicate, silicon nitride, silicon carbide, and glass, carbon, carbon-fiber, activated carbon, metal-oxide or metal networks and corresponding monoliths; or is encapsulated in a material (preferably silicon dioxide (SiO₂), phosphorus pentoxide (P₂O₅), magnesium oxide (MgO), chromium trioxide (Cr₂O₃), titanium oxide (TiO₂), zirconium oxide (ZrO₂) or alumina (Al₂O₃).

The preparation of a supported catalyst usable for low temperature oxy-dehydrogenation of ethane to ethylene is disclosed in the U.S. Pat. No. 4,596,787 A, 24 Jun. 1986 assigned to UNION CARBIDE CORP. A supported catalyst for the low temperature gas phase oxydehydrogenation of ethane to ethylene is prepared by (a) preparing a precursor solution having soluble and insoluble portions of metal compounds; (b) separating the soluble portion; (c) impregnating a catalyst support with the soluble portion and (d) activating the impregnated support to obtain the catalyst. The calcined catalyst has the composition MoₐV_{b}Nb_{c}Sb_{d}Xₑ. X is nothing or Li, Sc, Na, Be, Mg, Ca, Sr, Ba, Ti, Zr, Hf, Y, Ta, Cr, Fe, Co, Ni, Ce, La, Zn, Cd, Hg, Al, Tl, Pb, As, Bi, Te, U, Mn and/or W; a is 0.5-0.9, b is 0.1-0.4, c is 0.001-0.2, d is 0.001-0.1, e is 0.001-0.1 when X is an element.

Other examples of the low temperature oxy-dehydrogenation of ethane to ethylene using a calcined oxide catalyst containing molybdenum, vanadium, niobium and antimony are described in the U.S. Pat. No. 4,524,236 A, 18 Jun. 1985 and U.S. Pat. No. 4,250,346 A, 10 Feb. 1981, both assigned to UNION CARBIDE CORP. The calcined catalyst contains MoₐV_{b}Nb_{c}Sb_{d}Xₑ in the form of oxides. The catalyst is prepared from a solution of soluble compounds and/or complexes and/or compounds of each of the metals. The dried catalyst is calcined by heating at 220-550° C. in air or oxygen. The catalyst precursor solutions may be supported on to a support, e.g. silica, aluminium oxide, silicon carbide, zirconia, titania or mixtures of these. The selectivity to ethylene may be greater than 65% for a 50% conversion of ethane.

The above art teaches catalyst. None of the above art teaches or suggests the use of a continuous pulsed process in which oxygen and gaseous paraffin feeds are separated by a feed of an inert gas.

### SUMMARY OF THE INVENTION

The present invention provides a process for the oxidative dehydrogenation of one or more hydrocarbons selected from the group consisting of C₂₋₈ akanes and ethyl benzene to the corresponding C₂₋₈ alkene and styrene respectively comprising continuously sequentially pulsing an oxygen containing gas, one or more inert gases, said one or more hydrocarbons, and an inert gas through a catalytic oxidative dehydrogenation bed, either fixed, fluidized or moving, at a temperature from 300° C. to 700° C., a pressure from 0.5 to 100 psi (3.447 to 689.47 kPa) said catalytic oxidative dehydrogenation bed comprising at least one component capable of extracting oxygen from said oxygen containing gas while it passes through said bed and releasing oxygen to the oxidative dehydrogenation reaction while said one or more hydrocarbons passes through said bed, provided the pulse of said one or more inert gases is sufficiently long to provide a separation between said one or more hydrocarbons and said oxygen containing gas to prevent the formation of an explosive mixture of said hydrocarbon and said oxygen containing gas.

In a further embodiment said one or more C₂₋₈ alkanes and ethyl benzene is a single C₂₋₈ alkane and ethyl benzene having a purity of greater than 95%.

In a further embodiment the process has a productivity of not less than 1000 g of said C₂₋₈ alkene and styrene per kg of catalyst per hour.

In a further embodiment the process has a selectivity of not less than 95% to produce said C₂₋₈ alkene and styrene.

In a further embodiment the process has an hourly space velocity of said C₂₋₈ alkene and styrene of not less than 900 h⁻¹.

In a further embodiment the inert gas is selected from the group consisting of nitrogen, helium and argon and mixtures thereof.

In a further embodiment the oxygen containing gas is selected from the group consisting of oxygen, mixtures comprising from 30 to 70 wt % of oxygen and from 70 to 30 weight % of one or more inert gases, and air.

In a further embodiment said bed optionally further comprises a metal oxide.

In a further embodiment said C₂₋₈ alkane and ethyl benzene is a C₂₋₄ alkane.

In a further embodiment said bed comprises one or more catalysts selected from the group consisting of:

i) catalysts of the formula:

Ni_{f}AₐB_{b}D_{d}Oₑ

wherein
f is a number from 0.1 to 0.9 preferably from 0.3 to 0.9, most preferably from 0.5 to 0.85, most preferably 0.6 to 0.8;
a is a number from 0.04 to 0.9;
b is a number from 0 to 0.5;
d is a number from 0 to 0.0.5;
e is a number to satisfy the valence state of the catalyst;
A is selected from the group consisting Ti, Ta, V, Nb, Hf, W, Y, Zn, Zr, Si and Al or mixtures thereof;
B is selected from the group consisting of La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg and mixtures thereof;
D is selected from the group consisting of Ca, K, Mg, Li, Na, Sr, Ba, Cs, and Rb and mixtures thereof; and
O is oxygen; and
ii) catalysts of the formula:

MOᵢX_{g}Yₕ

wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg V, Ni, P, Pb, Sb, Si, Sn, Ti, U and mixtures thereof;
i=1;
g is 0 to 2;
h is 0 to 2, with the proviso that the total value of h for Co, Ni, Fe and mixtures thereof is less than 0.5;
(iii) a mixed oxide catalyst of the formula VₓMo_{y}Nb_{z}TeₘMeₙOₚ, wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.1 to 0.9;
y is from 0.001 to 0.5;
z is from 0.001 to 0.5;
m is from 0.001 to 0.5;
n is from 0.001 to 0.5; and
p is a number to satisfy the valence state of the mixed oxide catalyst.

In a further embodiment the oxidative dehydrogenation catalyst is supported on or admixed with inert matrix selected from oxides of titanium, zirconia, aluminum, magnesium, yttria, lantana, silica and their mixed compositions, to provide from 10 to 99 weight % of said catalyst and from 90 to 1 weight % of said oxides.

In a further embodiment said metal oxide is present in an amount to provide a weight ratio of metal oxide to supported oxidative dehydrogenation catalyst from 0.8:1 to 1:0.8 and said metal oxide is selected from the group consisting of NiO, Ce₂O₃, Fe₂O₃, TiO₂, Cr₂O₃, V₂O₅, WO₃, Al₂O₃ and ferrites of the formula MFeO₄ where M is selected from the group consisting of Mg, Mn, Co, Ni, Zn and Cd, and mixtures thereof.

In a further embodiment the C₂₋₄ alkane is ethane.

In a further embodiment the space-time yield of ethylene is not less than 1500 g/h per kg of catalyst.

In a further embodiment the catalyst has the formula VₓMo_{y}Nb_{z}TeₘMeₙOₚ, wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and:

x is from 0.2 to 0.5;
y is from 0.1 to 0.45;
z is from 0.1 to 0.45;
m is from 0.1 to 0.45;
n is from 0.01 to 0.45; and
p is a number to satisfy the valence state of the mixed oxide catalyst.

The foregoing embodiments may be combined in whole or part without deviating from the present invention or making a new invention.

### DETAILED DESCRIPTION

The catalyst useful in accordance with the present invention may be any catalyst suitable for the oxidative dehydrogenation of the hydrocarbon selected from the group consisting of C₂₋₈ alkane or ethyl benzene. In one embodiment the catalyst may be used in conjunction with a metallic oxide which takes oxygen from the oxygen containing gas (e.g. air) and then releases it to the oxidative dehydrogenation catalyst in the presence of the hydrocarbon. In another embodiment the catalyst itself is capable of taking oxygen from the oxygen containing gas either in the presence or absence of the metallic oxide, and using the oxygen in the oxidative dehydrogenation of the hydrocarbon.

The catalyst may comprise one or more catalyst selected from the group consisting of:

i) catalysts of the formula:

Ni_{f}AₐB_{b}D_{d}Oₑ

wherein
f is a number from 0.1 to 0.9 preferably from 0.3 to 0.9, most preferably from 0.5 to 0.85, most preferably 0.6 to 0.8;
a is a number from 0.04 to 0.9, preferably from 0.1 to 0.9;
b is a number from 0 to 0.5, preferably from 0.01 to 0.2;
d is a number from 0 to 0.05 preferably from 0.01 to 0.03;
e is a number to satisfy the valence state of the catalyst;
A is selected from the group consisting Ti, Ta, V, Nb, Hf, W, Y, Zn, Zr, Si and Al or mixtures thereof;
B is selected from the group consisting of La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg and mixtures thereof;
D is selected from the group consisting of Ca, K, Mg, Li, Na, Sr, Ba, Cs, and Rb and mixtures thereof; and
O is oxygen; and
ii) catalysts of the formula:

MoᵢX_{g}Yₕ

wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg V, Ni, P, Pb, Sb, Si, Sn, Ti, U and mixtures thereof;
i=1;
g is greater than 0 up to 2, preferably from 0.2 to 1.0;
h is greater than 0 up to 2, preferably from 0.2 to 1.0, with the proviso that the total value of h for Co, Ni, Fe and mixtures thereof is less than 0.5; and
(iii) a mixed oxide catalyst of the formula VₓMo_{y}Nb_{z}TeₘMeₙOₚ, wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.1 to 0.9, preferably from 0.2 to 0.5;
y is from 0.001 to 0.5, preferably from 0.1 to 0.45;
z is from 0.001 to 0.5, preferably from 0.1 to 0.45;
m is from 0.001 to 0.5, preferably from 0.1 to 0.45;
n is from 0.001 to 0.5, preferably from 0.01 to 0.45; and
p is a number to satisfy the valence state of the mixed oxide catalyst.

In one embodiment the catalyst is the catalyst of formula i) wherein f is from 0.5 to 0.85, a is from 0.15 to 0.5, b is less than 0.1 and d is less than 0.1. In catalyst i) typically A is selected from the group consisting of Ti, Ta, V, Nb, Hf, W, Zr, Si, Al and mixtures thereof, B is selected from the group consisting of La, Ce, Nd, Sb, Sn, Bi, Pb, Cr, Mn, Mo, Fe, Co, Cu, Ru, Rh, Pd, Pt, Ag, Cd, Os, Ir and mixtures thereof and D is selected from the group consisting of Ca, K, Mg, Li, Na, Ba, Cs, Rb and mixtures thereof.

In an alternative embodiment the catalyst is catalyst ii). In some embodiments of this aspect of the invention typically X is selected from the group consisting of Ba, Ca, Cr, Mn, Nb, Ti, Te, V, W and mixtures thereof, Y is selected from the group consisting of Bi, Ce, Co, Cu, Fe, K, Mg V, Ni, P, Pb, Sb, Sn, Ti and mixtures thereof.

In a further embodiment in the catalyst of formula (iii) the ratio of x:m is from 0.3 to 10, most preferably from 0.5 to 8, desirably from 0.5 to 6.

The methods of preparing the catalysts are known to those skilled in the art. For example for catalyst (iii), the active metal catalyst may be prepared by mixing aqueous solutions of soluble metal compounds such as hydroxides, sulphates, nitrates, halides lower (C₁₋₅) mono or di carboxylic acids and ammonium salts or the metal acid per se. For instance, the catalyst could be prepared by blending solutions such as ammonium metavanadate, niobium oxalate, ammonium molybdate, telluric acid etc. The resulting solution is then dried typically in air at 100-150° C. and calcined in a flow of inert gas such as those selected from the group consisting of N₂, He, Ar, Ne and mixtures thereof at 200-600° C., preferably at 300-500° C. The calcining step may take from 1 to 20, typically from 5 to 15 usually about 10 hours. The resulting oxide is a friable solid.

Typically the catalyst is supported. In the supported catalyst, the catalyst may be present in an amount from 1 to 95 preferably 10 to 95, most preferably from 30 to 80, desirably from 40 to 70 weight % of the supported catalyst and the support is present in an amount from 5 to 99 preferably from 90 to 5, most preferably from 70 to 20, desirably from 60 to 30 weight % of the total catalyst.

The support for the catalyst may be selected from the group consisting of porous silicon dioxide, fused silicon dioxide, kieselguhr, silica gel, porous and nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, yttrium oxide, aluminum silicate, silicon nitride, silicon carbide, and glass, carbon, carbon-fiber, activated carbon, metal-oxide or metal networks and corresponding monoliths; or is encapsulated in a material (preferably silicon dioxide (SiO₂), magnesium oxide (MgO), chromium trioxide (Cr₂O₃), titanium oxide (TiO₂), zirconium oxide (ZrO₂) or alumina (Al₂O₃).

Preferred supports include oxides of titanium, zirconium, aluminum, magnesium, yttrium, lanthanium, silicon and their mixed compositions or a carbon matrix.

It is also believed titanium silicates such as those disclosed in U.S. Pat. No. 4,853,202 issued Aug. 1, 1989 to Kuznicki, assigned to Engelhard Corporation, would be useful as supports in accordance with the present invention.

The support may have a broad range of surface area, typically greater than 25 m²/g up to 1,000 m²/g. High surface area supports may have a surface area greater than 250 m²/g (e.g. from 250 to 1,000 m²/g). Low to moderate surface area supports may have a surface area from 25 to 250 m²/g, preferably from about 50 to 200 m²/g. It is believed the higher surface area supports will produce more CO₂ during the oxidative dehydrogenation of the alkane.

The support will be porous and may have a pore volume up to about 5.0 ml/g, preferably less than 3 ml/g typically from about 0.1 to 1.5 ml/g, preferably from 0.15 to 1.0 ml/g.

It is important that the support be dried prior to use. Generally, the support may be heated at a temperature of at least 200° C. for up to 24 hours, typically at a temperature from 500° C. to 800° C. for about 2 to 20 hours, preferably 4 to 10 hours. The resulting support will be free of adsorbed water and should have a surface hydroxyl content from about 0.1 to 5 mmol/g of support, preferably from 0.5 to 3 mmol/g.

The amount of the hydroxyl groups in silica may be determined according to the method disclosed by J. B. Peri and A. L. Hensley, Jr., in J. Phys. Chem., 72 (8), 2926, 1968, the entire contents of which are incorporated herein by reference.

There are a number of methods which may be used to prepare the supported catalyst. The support could simply be impregnated with a solution or suspension of the catalyst. The catalyst would be dissolved or suspended in a solvent or diluent inert to the catalyst. The support would then be impregnated with the solution or suspension and dried, typically under an inert gas. The support and catalyst could also be spray dried.

In some instances the catalyst and the support may be combined and then comminuted to produce a fine particulate material having a particle size ranging from 1 to 100 micron. The communition process may be any conventional process including ball and bead mills, rotary, stirred and vibratory, bar or tube mills, hammer mills, and grinding discs. A preferred method of commnuition is a ball or bead mill.

In one embodiment of the invention the catalyst and the support are dry milled. It is also possible to wet mill the catalyst and support provided the resulting product is again dried and if necessary calcined.

The particulate material may be sieved if required to select the appropriate small particle size. The particulates may then be compacted and crushed to yield particles having a size from 0.1 to 1-2 mm. The particles or extrudates can be formed that can be further loaded in the catalytic reactor

The co-communition processes may be particularly useful relative to catalyst (iii).

The catalyst bed may also optionally further contain a metal oxide (different from the dehydrogenation catalyst) which takes up oxygen from a source such as pure oxygen gas or a mixture of oxygen containing gases or air and then supplies it to the catalyst for the oxidative dehydrogenation reaction in the presence of one or more hydrocarbons. The metal oxide may be selected from the group consisting of NiO, Ce₂O₃, Fe₂O₃, TiO₂, Cr₂O₃, V₂O₅, WO₃ and mixtures thereof and mixtures of NiO, Ce₂O₃, Fe₂O₃, TiO₂, Cr₂O₃, V₂O₅, WO₃ and mixtures thereof and aluminum in a weight ratio from 0.5:1 to 1:1.5 and ferrites of the formula MFeO₄ where, for example, M is Mg, Mn, Co, Ni, Zn or Cd and mixtures thereof and the weight ratio of oxidative dehydrogenation catalyst to metal oxide is from 0.8:1 to 1:0.8. In a further embodiment of the invention the metal oxide is a mixture of NiO, Ce₂O, Ce₂O₃, Fe₂O₃, TiO₂, Cr₂O₃, V₂O₅, WO₃ , rare earth oxides, ferrites of the formula M FeO₄ where, for example, M is selected from the group consisting of Mg, Mn, Co, Ni, Zn or Cd, and alumina in a weight ratio 0.8:1 to 1:0.8 and the oxidative dehydrogenation catalyst is used in an amount to provide a weight ratio of oxidative dehydrogenation catalyst to metal oxide from 0.8:1 to 1:0.8.

The feed to the reactor comprises four separate and sequential aliquots.

The one aliquot is an oxygen containing gas is selected from the group consisting of oxygen, mixtures comprising from 30 to 70 wt % of oxygen and from 70 to 30 weight % of one or more inert gases, and air. Some inert gases may be selected from the group consisting of selected from the group consisting of nitrogen, helium and argon and mixtures thereof. Preferably the oxygen containing gas is air as it provides for a much simpler plant operation.

One aliquot is an inert gas. The inert gas may be selected from those noted above.

One aliquot is a feed of one or more hydrocarbons selected from the group consisting of C₂₋₈ alkanes, and ethyl benzene, preferably a C₂₋₄ paraffin (i.e. ethane, propane, and butane). Preferably the feed is a single paraffin or ethyl benzene rather than a mixture of components. Most preferably the paraffin is ethane. The paraffin or ethyl benzene should have a purity greater than 90%, preferably greater than 95%, most preferably greater than 98%.

The final aliquot is an inert gas. The inert gas may be selected from those noted above.

The ratios of the gas components will be a function of the method of operating the reaction. The inert gas aliquot needs to be sufficiently large to separate the hydrocarbon stream from the oxygen containing stream as the components pass over the catalyst bed. The oxygen containing aliquot has to be large enough to provide sufficient oxygen to the catalyst and/or metal oxide to provide the oxygen needed for the oxidative dehydrogenation reaction when the hydrocarbon stream passes over the oxidative dehydrogenation catalyst bed optionally containing one or more metal oxides. One can calculate the ratio of oxygen to paraffin based on the stoichiometry of the reaction. However, the reaction will also be affected by the take up and release rate of the oxygen to and from the bed. In some cases it may be better not to completely deplete the oxidative dehydrogenation catalyst bed optionally containing a metal oxide of oxygen before recharging it. Typically the molar ratio of oxygen to hydrocarbon feed may range from 1:2.5 to 1:10, preferably from 1:2.5 to 1:3.5. In some cases it may be preferable to have smaller and more frequent succession of aliquots rather than larger aliquots and longer duration of the aliquot in the oxidative dehydrogenation catalyst bed optionally containing metal oxides. Given the foregoing one of ordinary skill in the art will be able to determine the preferred aliquot size and the frequency of the succession of aliquots (or cycle time).

A number of methods may be used to sequence, and size the aliquots of the gaseous feeds. There could be a series of valves to provide the various feeds to the inlet to the reactor. These valves would be controlled using for example a micro processor to deliver the appropriate amount and sequence of the feed gasses. One type of mechanical valve which might be use is a rotary valve similar to that disclosed in U.S. Pat. No. 3,779,712 issued Dec. 18, 1973 to Calvert et al., assigned to Union Carbide Corporation. Such a valve would not need the inert carrier gas for the particulate catalyst referred to in the disclosure as each of the feeds is already gaseous. An approach, when an aliquot of inert gas is between the hydrocarbon feed and the oxygen containing gas would be to use one rotor with four radially spaced apart passages (chambers) each about 90° from the other (two about 180° apart at the same radial distance from the center of the valve for the inert gas) and two about 180° apart at different distances radially from the center of the rotor from each other and the inlets for the inert gas. In this configuration only three feed lines to the rotor are required each at a radially different distance from the center of the rotor. Each turn of the rotor would provide an aliquot of hydrocarbon feed, an aliquot of oxygen containing gas buffered between two aliquots of inert gas. Other feed devices to sequence and size the aliquots for the feed would be apparent to those skilled in the art.

If only pulses of hydrocarbon and oxygen containing gas are used the rotor need only have two passages or chambers at a different radial location from the center of the rotor. However, in this configuration more care would be needed to ensure the mixture of gases in the reactor remains 25% outside of explosive mixtures.

If the above rotary valve approach is used the feed rate to the reactor is controlled by the speed of rotation of the rotor. The relative volumes of the components may be controlled by the relative sizes of the feed chambers or passages.

The oxidative dehydrogenation may be conducted at temperatures from 300° C. to 700° C., typically from 300° C. to 600° C., preferably from 350° C. to 500° C., at pressures from 0.5 to 100 psi (3.447 to 689.47 kPa), preferably from 15 to 50 psi (103.4 to 344.73 kPa), and the residence time of the paraffin in the reactor is typically from 2 to 30 seconds preferably from 5 to 20 seconds. The paraffin (alkane) may be a C₂₋₈, preferably a C₂₋₄ straight chained paraffin. The paraffin feed should be of purity of preferably 95%, most preferably 98% of the same paraffin. Preferably the paraffin is a high purity ethane. Preferably the process has a selectivity for the alkene or diene, preferably 1-alkene from the corresponding alkane of greater than 95%, preferably greater than 98%. The gas hourly space velocity (GHSV) will be from 900 to 18000 h⁻¹, preferably greater than 1000 h⁻¹. The space-time yield of alkene (e.g. ethylene) (productivity) in g/hour per Kg of catalyst should be not less than 900, preferably greater than 1500, most preferably greater than 3000, most desirably greater than 3500 at 350° C. It should be noted that the productivity of the catalyst will increase with increasing temperature.

The reactor may be a plug flow reactor.

The present invention will be demonstrated by the following non limiting examples.

### EXAMPLES

### Example 1

### Preparation of the Active Oxide Catalyst Phase no Support

2.65 g of ammonium heptamolybdate (tetrahydrate) and 0.575 g of telluric acid were dissolved in 19.5 g of distilled water at 80° C. Ammonium hydroxide (25% aqueous solution) is added to the Mo- and Te-containing solution at a pH of 7.5. Then water is evaporated under stirring at 80° C. The solid precipitate is dried at 90° C. 3.0 g of this precipitate is suspended in water (21.3 g) at 80° C. and 0.9 g of vanadyl sulfate and 1.039 g of niobium oxalate were added. The mixture was stirred for 10 min and then is transferred to the autoclave with a Teflon® (tetrafluoroethylene) lining. Air in the autoclave was substituted with argon, the autoclave was pressurized and heated to 175° C. and the system was kept for 60 hours at this temperature. Then the solid formed in the autoclave was filtered, washed with distilled water and dried at 80° C. The thus obtained active catalyst phase was calcined at 600° C. (2 h) in a flow of argon. The temperature was ramped from room temperature to 600° C. at 1.67° C./min. The powder was pressed then and the required mesh size particles were collected.

### Catalyst Activity

The catalyst was tested in oxidative dehydrogenation of ethane using a gas mixture O₂/C₂H₆ with an O₂ content of 25% outside the explosive limit. The mixture was fed in the plug-flow reactor with the gas hourly space velocity of 900 h⁻¹ at a pressure of 1 atm.

The catalyst was tested at 420° C., the catalyst loading 0.13-1.3 g; fraction 0.25-0.5 mm, a flow type reactor with a stationary catalyst bed was used. The catalyst was heated to 360° C. in the reaction mixture and the catalytic activity was measured at 420° C. The data for are presented in the Table 1 (Entry 1).

### Example 2

The catalyst of Example 1 was placed in the same reactor used in Example 1 (0.13 g) and was tested in oxidative dehydrogenation of ethane under conditions of periodic regimes by varying the space velocity and duration of the stages as set out in Table 1.

Table 1 shows the catalytic performance of the V—Mo—Nb—Te oxide catalyst in oxidative dehydrogenation of ethane in conventional mode (direct oxidation, 75% ethane and 25% oxygen) and in a periodical mode (separate flows of pure ethane and air).

TABLE 1

| | | Space-time yield | |
|---|---|---|---|
| | | of ethylene | |
| | Space Velocity | (productivity) g/hr | Selectivity to |
| Example | (VHSV) h⁻¹ | per 1 kg of catalyst | ethylene % |
| 1 (comparative) | 900 | 210 | 90-92 |
| 2 | 900 | 980 | 96 |
| | 3,000 | 1,800 | 97 |
| | 10,000 | 3,500 | 98 |

It is seen from this comparison that the process of the invention, periodical mode, provides at least 2-3 time higher productivity of the same unoptimized catalyst in the oxidative dehydrogenation of ethane. The use of pure ethane contributes to the higher space velocity. Also the selectivity remains high, >95%, without a clear dependence on the space velocity of the gas flows of ethane and air. Air can be used as the source for oxygen without the need for separation and purification of oxygen and the separation of oxygen at the reactor outlet. This eliminates two significant capital costs from a plant to practice the process. The process is safe, as explosive limits are not approached, and energy and resource efficient.

What is claimed is:

1. A process for the oxidative dehydrogenation of one or more hydrocarbons selected from the group consisting of C₂₋₈ akanes and ethyl benzene to the corresponding C₂₋₈ alkene and styrene respectively comprising continuously sequentially pulsing an oxygen containing gas, one or more inert gases, said one or more hydrocarbons and one or more inert gases through a catalytic oxidative dehydrogenation bed, either fixed, fluidized or moving, at a temperature from 300° C. to 700° C., a pressure from 0.5 to 100 psi (3.447 to 689.47 kPa) said catalytic oxidative dehydrogenation bed comprising at least one component capable of extracting oxygen from said oxygen containing gas while it passes through said bed and releasing oxygen to the oxidative dehydrogenation reaction while said one or more hydrocarbons passes through said bed, provided the pulse of said one or more inert gases is sufficiently long to provide a separation between said one or more hydrocarbons and said oxygen containing gas to prevent the formation of an explosive mixture of said hydrocarbon and said oxygen containing gas.

2. The process according to claim 1, wherein said one or more C₂₋₈ alkanes and ethyl benzene is a single C₂₋₈ alkane and ethyl benzene having a purity of greater than 95%.

3. The process according to claim 2, having a productivity of not less than 1000 g of said C₂₋₈ alkene and styrene per kg of catalyst per hour.

4. The process according to claim 3, having a selectivity of not less than 95% to produce said C₂₋₈ alkene and styrene.

5. The process according to claim 4, having an hourly space velocity of said C₂₋₈ alkene and styrene of not less than 900 h⁻¹.

6. The process according to claim 5, wherein the inert gas is selected from the group consisting of nitrogen, helium and argon and mixtures thereof.

7. The process according to claim 6, wherein the oxygen containing gas is selected from the group consisting of oxygen, mixtures comprising from 30 to 70 wt % of oxygen and from 70 to 30 weight % of one or more inert gases and air.

8. The process according to claim 7, wherein said bed optionally further comprises a metal oxide.

9. The process according to claim 8, wherein said C₂₋₈ alkane and ethyl benzene is a C₂₋₄ alkane.

10. The process according to claim 9, wherein said bed comprises one or more catalysts selected from the group consisting of:
i) catalysts of the formula:
Ni_{f}AₐB_{b}D_{d}Oₑ
wherein
f is a number from 0.1 to 0.9 preferably from 0.3 to 0.9, most preferably from 0.5 to 0.85, most preferably 0.6 to 0.8;
a is a number from 0.04 to 0.9;
b is a number from 0 to 0.5;
d is a number from 0 to 0.0.5;
e is a number to satisfy the valence state of the catalyst;
A is selected from the group consisting Ti, Ta, V, Nb, Hf, W, Y, Zn, Zr, Si and Al or mixtures thereof;
B is selected from the group consisting of La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg and mixtures thereof;
D is selected from the group consisting of Ca, K, Mg, Li, Na, Sr, Ba, Cs, and Rb and mixtures thereof; and
O is oxygen; and
ii) catalysts of the formula:
MoᵢX_{g}Yₕ
wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg V, Ni, P, Pb, Sb, Si, Sn, Ti, U and mixtures thereof;
i=1;
g is 0 to 2;
h is 0 to 2, with the proviso that the total value of h for Co, Ni, Fe and mixtures thereof is less than 0.5;
(iii) a mixed oxide catalyst of the formula VₓMo_{y}Nb_{z}TeₘMeₙOₚ, wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.1 to 0.9;
y is from 0.001 to 0.5;
z is from 0.001 to 0.5;
m is from 0.001 to 0.5;
n is from 0.001 to 0.5; and
p is a number to satisfy the valence state of the mixed oxide catalyst.

11. The process according to claim 10, wherein the oxidative dehydrogenation catalyst is supported on or admixed with inert matrix selected from oxides of titanium, zirconia, aluminum, magnesium, yttria, lantana, silica and their mixed compositions, to provide from 10 to 99 weight % of said catalyst and from 90 to 1 weight % of said oxides.

12. The process according to claim 11, wherein said metal oxide is present in an amount to provide a weight ratio of metal oxide to supported oxidative dehydrogenation catalyst from 0.8:1 to 1:0.8 and said metal oxide is selected from the group consisting of NiO, Ce₂O₃, Fe₂O₃, TiO₂, Cr₂O₃, V₂O₅, WO₃, Al₂O₃ and ferrites of the formula MFeO₄ where M is selected from the group consisting of Mg, Mn, Co, Ni, Zn and Cd, and mixtures thereof.

13. The process according to claim 12, wherein the C₂₋₄ alkane is ethane.

14. The process according to claim 13, wherein the space-time yield of ethylene is not less than 1500 g/h per kg of catalyst.

15. The process according to claim 14, wherein the catalyst has the formula VₓMo_{y}Nb_{z}TeₘMeₙOₚ, wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.2 to 0.5;
y is from 0.1 to 0.45;
z is from 0.1 to 0.45;
m is from 0.1 to 0.45;
n is from 0.01 to 0.45;
and p is a number to satisfy the valence state of the mixed oxide catalyst.
